(19) United States
(12) Patent Application Publication
(10) Pub. No.: US 2011/0000484 A1
(43) Pub. Date: Jan. 6, 2011

(54) VASCULAR THERAPY USING NEGATIVE PRESSURE

(75) Inventor: Jeffry S. Melsheimer, Springville, IN (US)

Correspondence Address: Buchanan Nipper LLC, P.O. Box 700, Perrysburg, OH, 43552, US

(73) Assignee: Cook Incorporated, Bloomington, IN (US)

(21) Appl. No.: 12/497,252

(22) Filed: Jul. 2, 2009

(51) Int. Cl. A61B 17/00

(52) U.S. Cl. 128/202.12

(57) ABSTRACT

System and methods for applying vascular therapy to a body vessel using a chamber capable of negative and/or positive pressure relative to ambient are provided. The chamber includes one or more pressure-isolated chambers. A pressure and/or vacuum source is connected to the pressure chamber, and is configured to provide distinct pressures within each pressure-isolated chamber. A controller is coupled to the pressure source, and is configured to control the pressure source such that pressure within each of the pressure-isolated chambers is controlled cyclically to simulate a pulsating pump or peristaltic-like pump action within the body vessel. The use of negative pressure is sequenced such that the resistance to pressure toward the heart is reduced. This effectively “pulls” blood flow toward the heart and creates more space for incoming blood flow. During simulation, medical devices may be introduced to and/or diagnostics may be performed on the targeted vessel.

### BACKGROUND

The present embodiments invention generally relate to methods and systems for enhancing therapeutic treatment of various vascular diseases using a negative and/or positive pressure atmosphere. In particular, they relate to an apparatus and method for surrounding a body part with a hypobaric chamber and applying various vascular treatments while the hypobaric chamber is being operated at negative pressure atmosphere.

There are many patients who suffer from diminished blood flow through the intravascular system of the human body. Causes for such diminished blood flow include diabetes mellitus, frost bite, burn victims, venous diseases, and others. Diminished blood flow in respective parts of the human body lead to such problems as pain, slower healing, breakdown of soft tissue, under-effective valves, varicosities, and even eventual tissue loss.

Treatments for diminished blood flow through the intravascular system include therapeutic agents (blood thinner), compression techniques such as compression stockings that apply a positive pressure and intimate contact with the body, venoplasty, vessel removal, valvuloplasty, prosthetic valves or stents. As to venous diseases, compression techniques, in particular, can support the weaker veins in the legs and assist the action of the calf pump action in returning venous blood to the trunk. Yet, the compression techniques are often uncomfortable and obtrusive, i.e., interfering with other types of therapies. Compression techniques can also be erosive or traumatic to the skin. In this instance, patients with sensitive skin or damaged skin, such as a burn victim, can lead to further damage to the skin.

Thus, there remains a need for methods and systems for enhancing treatment of various vascular diseases while minimizing the interface between the skin and system.

### SUMMARY

Accordingly, provided are a system and methods for vascular therapy using at least negative pressure. In particular, the system and methods are ideally used for venous therapy while the pressure and/or hypobaric chamber, using negative pressure, is configured to simulate pulsating pump action, like calf pump action, or peristaltic-like pump action within a body vessel such as a vein. The pressure chamber is configured to minimize contact with the body part, which is particularly useful for patients having sensitive skin and/or patients suffering from burn wounds.

Preferably, the use of negative pressure, especially in multiple chambers, is sequenced such that the pressure downstream or toward the heart is reduced. This should “pull” or induce blood flow toward the heart by reducing the pressure resistance load the blood must overcome, and create more space for incoming blood flow. Preferably, the “pull” should be downstream of the source of resistance, such as stenosis, obstructions, faulty valves, or the like. This beneficially avoids applying a positive pressure at or upstream the source of the resistance, such as thrombosis, which causes a further increase in resistance.

In one embodiment, the pressure and/or hypobaric chamber includes one or more pressure-isolated chambers having individually controlled pressures within each chamber. The pressure chamber can include an inlet port configured to receive the body part and a cuff disposed at the inlet port to sealably engage with the body part. The pressure chamber can be coupled to one or more pressure sources configured to apply a negative and/or positive pressure within the chamber. A controller can be coupled to the pressure source and configured to regulate the pressure within the chamber so that pulsating pump action and/or peristaltic-like pump action is simulated within the body vessel. The controller may also be configured to control the pressure source such that the pressure within each of the pressure-isolated chambers is controlled cyclically to simulate the pulsating pump and/or peristaltic-like pump action within the body vessel. The controller may be further configured to control the pressure source such that the pressure within one of the first and second chambers is decreased to a negative pressure relative to ambient and the pressure within the other one of the first and second chambers is maintained to a pressure greater than the negative pressure.

One or more glove apparatuses can be disposed within the chamber for accessing the portion of the body that is within the chamber from external. The glove apparatuses permit the clinician to perform procedures, such as injection of therapeutic agents, within the chamber during operation of the chamber. An outlet port configured to receive the body part such that a portion thereof extends outwardly past the outlet port can be included. This allows the clinician to focus on certain portions of the body part that need the treatment. A cuff can be disposed at the outlet port to sealably engage with the body part. Optionally, an impermeable article can be disposed at the outlet port, the impermeable article adapted to receive and contain the extended portion of the body part. The impermeable article can be sized to reduce the area of contact against the skin.

In one aspect, a method of treating a body vessel within a body part with a medical device is provided. One step includes positioning a pressure or hypobaric chamber around a body part of a patient. Another step can include sealing the body part within the chamber such that the pressure differential within the chamber can be maintained. Another step can include simulating pulsating pump action and/or peristaltic-like pump action by varying the pressure within the chamber from a negative pressure relative to ambient to a pressure greater than the negative pressure. For example, the pressure can be ambient or even a positive pressure relative to ambient.

While simulating pulsating pump action and/or peristaltic-like pump action, vascular or venous system diagnostic/investigation and/or therapy can be applied. For example, during simulation, and especially during the dilation of the vessel caused by a negative pressure environment, the medical device can be introduced and/or navigated more easily into the body vessel. The medical device can include guide wires, catheters, atherectomy devices, filters, occluders, stents, valves, balloons, perfusion devices, or other devices commonly introduced intravascularly. The medical device can also include a bioactive, wherein the pressure of chamber can aid in eluting the bioactive more efficiently.

Another step can include imaging the body vessel with an imaging device to characterize the condition of the body vessel before and/or during simulation of pulsating pump action and/or peristaltic-like pump action for diagnostics and/or investigation of the vessel. Types of imaging devices include at least one of ring magnets, lenses, ultrasonic transducers, fluoroscopy, x-rays or other interventional radiological devices and systems. Examples of diagnostics/investigations include diagnosing deep venous thrombosis, distinguishing blood clots from obstructions, seeing the working of the deep leg vein valves, evaluating congenital vein problems, identifying a vein for bypass grafting, conditions and characteristics of valves, and identifying narrowing veins. Techniques for diagnostics/investigations can include venography (ascending/descending venography), plethysmography, duplex ultrasonography (doppler, duplex scanning), and/or others known in the art.

In another aspect, a method of applying venous therapy to a body vessel within a body part using a multiple chamber pressure or hypobaric chamber including one or more pressure-isolated chambers having individually controlled pressures within each chamber is provided. In this instance, the pressure within one chamber of a first and a second chamber can be decreased to a negative pressure relative to ambient, while the pressure within another chamber of the first and second chambers can be maintained to a pressure greater than the negative pressure. Preferably, the pressure is decreased for a time period in only one of the chambers while the other chambers are at a greater pressure. After passage of the time period, that chamber's pressure is increased to a greater pressure, while the next chamber's pressure is decreased to a negative pressure for a time period. The time period can be same for all chambers or can vary depending on the therapy. The negative pressure of the chambers can be timely sequenced such that the pump action is maintained or can be timely synchronized with a pulse or heartbeat.

Optionally, the pressure chamber includes a first chamber, a second chamber, and a third chamber, where the first chamber is positioned distal to the second chamber and each distal to the third chamber. The pressure within each of the chambers can be controlled cyclically to simulate a peristaltic-like pump action within the body vessel. For example, the pressure within one of the first, second, and third chambers can be decreased to a negative pressure relative to ambient. Then, the pressure within another of the first, second, and third chambers can be decreased to a negative pressure relative to ambient. Lastly, the pressure within the last of the first, second, and third chambers can be decreased to a negative pressure relative to ambient. During each of the decreasing steps, the pressure within the other two chambers of the first, second, and third chambers can be maintained at a pressure greater than the negative pressure of each chamber. Optionally, a first pair, or set if more than three chambers are present, of the first, second, and third chambers can be decreased to a negative pressure relative to ambient. Then, the pressure within a second pair or set of the first, second, and third chambers can be decreased to a negative pressure relative to ambient. Lastly, the pressure within a third pair or set of the first, second, and third chambers can be decreased to a negative pressure relative to ambient.

Furthermore, the pressure within each chamber can be controlled to affect the pressure, flow and direction of blood within the vessel. This can be beneficial for investigation and/or diagnostics of the vessel to identify fault valves, obstructions, etc. For example, the pressure within each chamber can be controlled such that blood pressure is varied, such as at a higher or more intense pressure; blood flow is varied, such as at a higher or lower rate or even stopped; direction of blood pressure is varied, such as to flow opposite in an opposite direction. This is particularly useful with fluoroscopy where the effects of the imageable or contrast dye can be observed.

The above, as well as other advantages of the present invention, will become readily apparent to those skilled in the art from the following detailed description of a preferred embodiment when considered in the light of the accompanying drawings.

### BRIEF DESCRIPTION OF THE DRAWINGS

FIG. 1 is a perspective view of a pressure or hypobaric chamber.

FIG. 2 is a schematic depicting a system controller connected to a vacuum source and a pressure or hypobaric chamber.

FIG. 3 is a perspective view of a pressure or hypobaric chamber including glove apparatuses.

FIG. 4 depicts an air lock attached to a side of the chamber in FIG. 3.

FIG. 5 is a perspective view of a pressure or hypobaric chamber having a plurality of pressure-isolated chambers.

FIG. 6A is graphical representation of peristaltic-like pump cycle within an embodiment of a pressure or hypobaric chamber.

FIG. 6B is depicts dilation of the vessel according to the peristaltic-like pump cycle of FIG. 6A.

### DESCRIPTION OF PREFERRED EMBODIMENTS

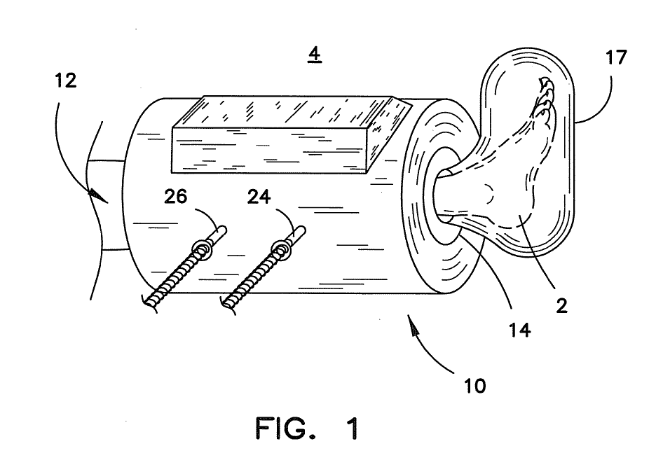

With reference to FIG. 1, a leg of a patient is shown inserted within a pressure or hypobaric chamber 10. It is to be understood that any body part can be inserted within the chamber 10 for treatment. The chamber 10 is sized and shaped appropriately to surround a portion of the body part 2. The chamber 10 can include one or more ports for insertion and exiting of the body part 2. For example, two ports 12, 14 may be ideal for situations in which to focus treatment to a certain area of the body, without performing the treatment unnecessarily to other parts of the body.

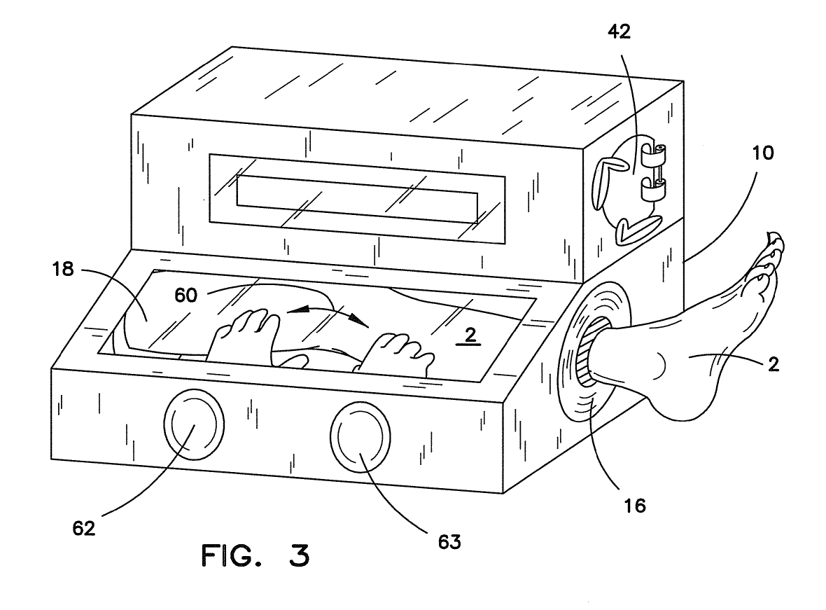

A cuff 16 can be included at one or more of the ports 12, 14, as shown in FIG. 3. The cuff is sized and shaped to surround the portion of the body part 2 in a minimally invasive manner. That is, there are some situations when it is undesirable to contact large areas of skin or to apply a positive pressure around the skin. For example, when treating burn victims, although the chamber 10 under a negative pressure can help dilate blood vessels contained within the chamber 10, applying tightened cuffs or constant-pressurized cuffs at the burnt skin can have adverse affects like constriction or inhibiting blood flow. The cuffs 16 may be pliable and/or elastic where insertion of the body part 2 would slightly expand the center of the cuffs 16 in order to achieve a sealable coupling. The sealable coupling is sufficient to allow a negative and/or positive pressure differential relative to outside ambient 4 to be maintained. Optionally, the cuffs 16 may be inflatable with a positive pressure to achieve a sealable coupling with the body part.

With reference to FIG. 1, an impermeable article 17, such as a glove or sock or other such article having a cavity therethrough with an open end and closed end, may be used on the distal portion of the limb extending beyond chamber 10. The term “impermeable” is used to describe the ability of an object to maintain pressure differential relative to ambient within its cavity. The impermeable article 17 is configured to surround the extended body part and maintain the pressure differential between inside the article and ambient. The article can be made of rubber or other like material. The article 17 can be used in conjunction with, or would replace the distal cuff, and offer a less traumatic interface between the article and body part which would be beneficial to patients with sensitive skin or burn victims. The cavity of the impermeable article may be sized such that a gap is created to minimize contact with the body part.

The chamber 10 is preferably made of a light weight material having sufficient strength to withstand the negative pressure and/or positive pressure changes and/or light enough for portability to allow the operator to move the chamber 10 via a cart or other like movable means to a position near the patient. Once positioned, the chamber 10 can then slide from the cart to a position where the chamber can receive the body part 2.

According to FIG. 3, in order to allow observation of the body part 2 and/or instruments during treatment, the chamber 10 can also have one or more viewing ports 18 or windows. In some embodiments, the chamber 10, or viewing port 18, may be made entirely of transparent material, such as acrylic or polycarbonate sheeting or Lexan®, which is known to be durable, light weight and cleanable. Polyvinylcarbonate (PVC) (static-dissipative or nondissipative) and polypropylene or other polymer or plastics can also be used. Stainless steel, aluminum, or other metals may also be used in construction of the chamber.

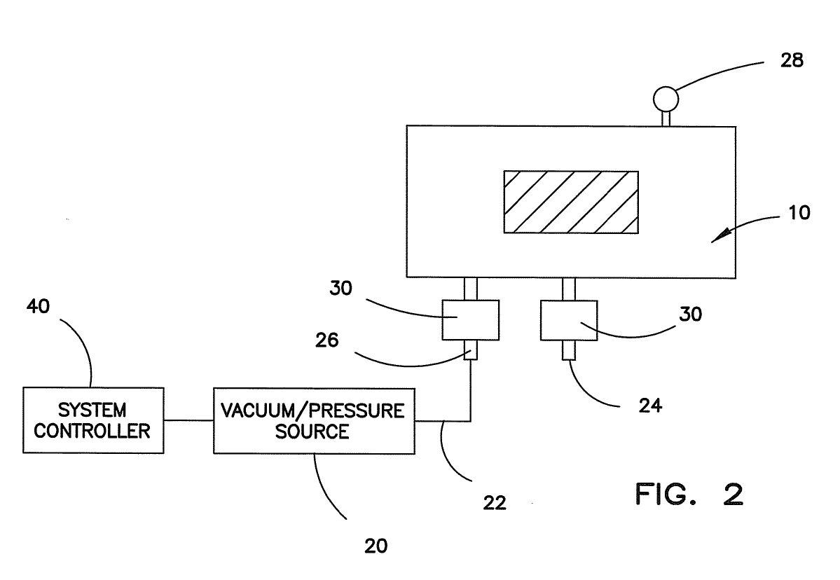

As is shown in FIG. 2, the chamber 10 can include one or more pressure or vacuum sources 20 configured to produce a negative and/or positive pressure within the chamber. Multiple pressure sources for negative and/or positive pressure can be coupled to the chamber. The pressure source 20 can include a pump, fan blower, or other means known in the art. The pressure source 20 can be attached to the chamber 10. Optionally, the pressure source 20 can be external to the chamber 10 and coupled to the chamber via a conduit means 22, such as tubing, pipes, hoses or the like by clamps or fittings or other connective means, as shown in FIG. 2. In this embodiment, the chamber 10 can also include an air inlet port 24 and an exhaust port 26, each of which can be connected with the conduit means 22. The air inlet port 24 and the exhaust port 26 can be used singularly or in conjunction to allow the chamber 10 maintain a pressure differential relative to ambient. To filter or purify the air, the air inlet port may also include a filter, such as a charcoal filter, HEPA filter, ULPA filter, or the like.

As illustrated in FIG. 2, the chamber 10 can include a pressure gage 28 to indicate the amount of pressure within the chamber 10 and/or a pressure valve 30, such as a solenoid valve, to regulate the flow of air between the pressure source 20 and the chamber 10. The pressure valve 30 can be proportionally controlled so that the pressure within the chamber 10 is sufficient for treatment but not so great that it results in discomfort for the patient. After operation, the pressure valve 30 can be fully open in order to balance the ambient pressure and the chamber 10 for permitting easy removal of the body part from the chamber 10. A pressure valve can also be included on the air inlet port 24 and/or the exhaust port 26. Further, the inlet and exhaust ports may be capped or plugged with a plug.

To regulate the conditions of the chamber 10, the chamber 10 can also include sensors that are electrically and/or pneumatically coupled to a system controller 40, as shown in FIG. 2. The system controller 40 is also electrically and/or pneumatically coupled to the pressure source 20. Examples of sensors can include a pressure transducer, a temperature sensor, a humidity sensor, and sensors for pulse rate, blood pressure rate, heart rate, ankle-brachial index, among others known in the art. The system controller 40 may include a computer having at least one processor or CPU and inputs. Instructions can be stored in memory, including random access memory (RAM) and read-only memory (ROM), which can be coupled to the CPU. Instructions are executed by the CPU to control and make decisions for the pressure source and the pressure chamber and other components in form of outputs to direct, monitor, and otherwise functionally cooperate with the components. The outputs can be fully automatic without any operator input and/or manually controlled by the operator. The controller can be configured as a programmable logic controller (PLC). The inflation of the cuff, if inflatable, may also be regulated by the pressure source 20 and controller 40. In addition, to regulate the humidity within the chamber, a humidification/dehumidification module can be connected to the chamber. Likewise, to control the temperature, a refrigeration module and/or heating module can be connected to the chamber. The heating module can include an electric or gas heater. Control of the temperature and/or humidity within the chamber 10 can be advantageous in applications requiring cuffs or inflatable cuffs because of the intimate contact with skin can cause discomfort caused by sweating and heating and decrease the sealability of the cuff.

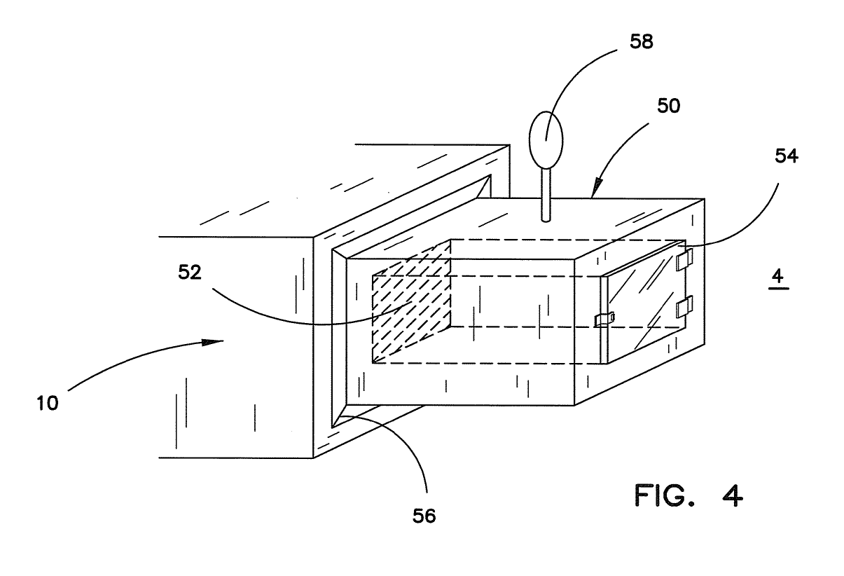

With reference to FIG. 3, it is often desirable to pass instruments, parts, and/or other devices in-and-out of the inside the chamber 10. One or more doors 42 can be provided to permit access within the chamber 10. The doors can be attached to the chamber using conventional means known in the art, such as via hinges and a latch, as shown in FIG. 4. In certain applications it is desirable to maintain the pressure within the chamber 10. Accordingly, one or more air-locks 50 may be included in the chamber 10, as shown in FIG. 4. The air-lock 50 is an intermediate chamber designed such that a chamber end 52 of the air-lock 50 is in communication with the chamber 10 and an ambient end 54 of the air-lock 50 is in communication with ambient 4. Both ends 52, 54 of the air-lock 50 can have sealable doors in order to decrease the amount of loss of chamber pressure from the chamber 10 and/or decrease the amount of gain of ambient pressure within the chamber 10. The air-lock 50 can be mounted on a side of the chamber 10 to allow for a means for pass-through of instruments, devices, and/or parts into and out of the chamber 10. The air-lock 50 may include a flange 56 and/or a gasket for enhancing sealing performance between the air-lock 50 and the chamber 10. A pressure gage 58 can be coupled to the air-lock 50 to indicate the amount of pressure therein. Capped ports can be included in the air-lock 50 for purging the air-lock with a gas, such as nitrogen. The air-lock 50 can be made of identical materials of the chamber 10 or different materials selected from materials listed above.

Neutral or constant pressure gloves 60 may also be included with the chamber 10 at access ports 62, 63, as shown in FIG. 3. Preferably, the gloves 60 are positioned and oriented such that the clinician can access the pertinent body part 2 within the chamber 10 in order to perform various treatments. For example, the clinician can use a syringe for injecting therapeutic or imageable agents, such as injecting scleroscent transdermally to ablate varicose veins, for massaging or manipulating the body part for blood flow, or the like. The gloves 60 extend within the chamber 10 and access to the gloves can be achieved by placing hands through access ports 62, 63. The gloves 60 are sealably attached to the chamber 10 such that no ambient air enters into the chamber. The gloves 60 can be designed to maintain its shape and functionality under negative and/or positive pressure environments, yet can have sufficient flexibility in order for the clinician to grasp for instruments and/or devices or to apply pressure or contact to the body part 2. Accordingly, the gloves 60 can be configured to prevent excess negative pressure to the clinician's hands. The life of the gloves is also extended as excessive flexing of gloves is known to deteriorate the life of the gloves. Further, sleeves, straight or accordion-shaped, can be attached to the gloves 60 for those applications requiring arm movement. Preferably, the chamber 10 includes both the gloves 60 and the air-lock 50.

With references to all of the figures, the pressure within chamber 10 can be decreased to a negative pressure sufficient to cause the body vessels within the body part to dilate for treatment thereof. Generally, the amount of vacuum or negative pressure within the chamber 10 for effective treatment can vary depending on the type of treatment and/or duration of treatment. For example, a negative pressure up to about 30 inches (Hg) below ambient is capable, although it is understood that one skilled in the art would select the suitable negative pressure for optimal treatment, such as lower negative pressures of about 1 inch (Hg) or less to about 10 inches (Hg). Dilation of body vessels within the chamber 10 can allow an increase of blood flow therethrough. Dilation of body vessels within the chamber 10 can also permit easier navigation of medical devices, such as guide wires, catheters, atherectomy devices, or the like, through the body vessel. For treatment of clots, after implantation of a filter, such as a venous filter, dilation of blood vessels can help dislodged clots. In addition, other medical devices can be used to dislodge clots while dilation of the body vessels with the chamber 10.

It may also be desirable for the chamber 10 to have imaging components configured to facilitate imaging of venous therapy of dilated body vessels within the chamber. The chamber may be used in conjunction with imaging components including at least one of ring magnets, lenses, ultrasonic transducers, fluoroscopy, x-rays or other interventional radiological devices and systems know in the art to be used for imaging. The type of imaging component may influence the selection of material for the chamber and placement of the imaging component. For example, an ultrasound device is preferably placed inside the chamber in order to avoid transmission interferences by the wall of the chamber. Similarly, a metal chamber may interfere with the imaging of the ring magnet. Each of the devices is connected to the system controller 40 or a group of controllers which are programmed to perform certain investigations. Examples of investigations include diagnosing deep venous thrombosis, distinguishing blood clots from obstructions, seeing the working of the deep leg vein valves, evaluating congenital vein problems, identifying a vein for bypass grafting, conditions and characteristics of valves, and identifying narrowing veins.

For example, the imaging components can be used to perform venography (ascending/descending venography), plethysmography, and/or duplex ultrasonography (doppler, duplex scanning). Venography (also called phlebography) is a procedure in which an x-ray of the veins, a venogram, is taken after a contrast dye is injected into the veins. The contrast dye permits the clinician to evaluate the size and condition of the veins, for example, to locate the presences of the deep vein thrombosis (ascending venography) and/or evaluate the function of the deep vein valves (descending venography). Duplex ultrasonography incorporates two elements which are displayed on the same screen (duplex) to facilitate investigation: b-mode, pulsed-doppler display to visualize the blood flow within the dilated vessel; and color-doppler display to visualize the structure and hemodynamics within the dilated vessel. Plethysmography is a test measuring blood volume in the lower leg due to temporary venous obstruction. The test is performed by inflating a pneumatic cuff with positive pressure around the thigh to sufficient pressure to cut off venous flow but not arterial flow. This causes the venous blood pressure to rise until it equals the pressure under the cuff. When the pressure within the cuff is released, the normal response will include a rapid venous runoff and a prompt return to the resting blood volume. However, if there is a delay in venous runoff and/or return to resting blood volume, venous thrombosis more than likely has altered the normal response to temporary venous obstruction. In addition, venous thrombosis also alters the increase in blood volume after cuff inflation.

A portion of the chamber 10 in the figures can be placed on the body part to induce pulsations within a vessel; for example, around a calf in order to simulate calf pump action. Negative pressure can be timely sequenced such that a pulsating pump action is maintained to dilate the body vessel cyclically in order to avoid adverse conditions such as edema. The sequence can be also coordinated and/or synchronized with the pulse/heartbeat through use of sensors and the controller. For example, a sensor for sensing the blood pulse can be provided at the ankle so that when the pulse is sensed the portion of the chamber 10 has a negative pressure simultaneously with, or slightly delayed based on, the pulse.

It can be advantageous to simulate pump action for a patient who is stationary in order for investigation of the patent's vascular or venous system to occur. In one example, venous valves in the deep vein of a leg can be observed during simulated pump action. For instance, leg vein valve action can be observed while using the chamber during simulation of a calf pump action in vessels that are being investigated with fluoroscopy. Using calf pump action can aid the valves in the pushing of blood flow through the venous system.

In another example, plethysmography can be performed while the body part 2 is inserted into the chamber 10. Here, the cuff 16 at the inlet can be inflatable to apply a positive pressure at the portion of the body part which is in contact with the cuff. The chamber 10 can operate having a negative pressure, ambient or a positive pressure while plethysmography is being performed as described above.

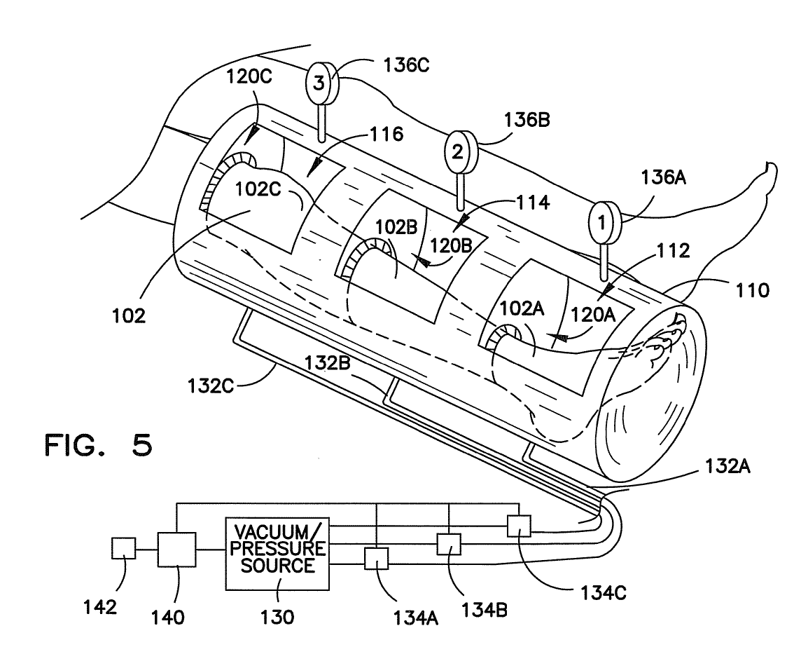

FIG. 5 illustrates another embodiment of the chamber 110 including a plurality of isolated pressure chambers, shown as a first isolated pressure chamber 112, a second isolated pressure chamber 114, and a third isolated pressure chamber 116, although two or four or more isolated pressure chambers could be used. The chamber 110 can have one inlet for receiving the body part as the one end can be closed or capped. The first isolated pressure chamber 112 is shown positioned around the foot 102A, the second isolated pressure chamber 114 is shown positioned around the distal calf pump region 102B of the calf, and the third isolated pressure chamber 116 is shown positioned around the proximal calf pump region 102C of the calf.

Each isolated pressure chamber 112, 114, 116 can be separated from one another by cuffs 120 or bulkheads that sealably engage the body part 102 such that a different negative or positive pressure can be maintained within each of the isolated pressure chambers. This configuration can be particularly useful to provide peristaltic-like pump action for treatment alone or during venous therapy. The term “peristaltic-like pump action” is used to describe using rhythmic dilations to allow bodily fluid to propel in a desired direction. It can be advantageous to simulate peristaltic-like pump action for a patient who is stationary in order for investigation of the patent's vascular or venous system to occur. For example, leg vein valve action can be observed while using the chamber 110 to simulate circulation that is either more intense or at a higher blood pressure, or that is taking place at a higher rate or blood rate, or both. The chamber 110 may be used to produce momentarily a retrograde blood flow or even a stoppage of blood flow, and also used to stall the return of blood flow so that contrast will linger in vessels that are being investigated with fluoroscopy. Using peristaltic-like pump action can essentially pull/push blood flow through the venous system in either direction, regardless of the presence of valves or obstructions. This treatment can help force back blood flow toward the heart which is particularly beneficial to patients without any valves or with faulty valves. In another application, peristaltic-like pump action can simulate venous blood flow during exercising so that the venous system and valves can be observed, even though the patient is stationary.

As shown in FIG. 5, each isolated pressure chamber is coupled to a pressure and/or vacuum source 130 via a conduit means 132A, 132B, 132C, examples described above. A pressure valve 134A, 134B, 134C, such as a solenoid valve, can be coupled between each isolated pressure chamber 112, 114, 116 and the pressure source 130. Valves 134A, 134B, 134C are controlled in sequence by separate electrical pulse signals from the respective outputs of the system controller 140 working in conjunction with a pulse generator 142. Also, a pressure relief valve can be coupled to each isolated pressure chamber in order to control the rate of ambient pressure entering into the chambers. Pressure gages 136A, 136B, 136C can be coupled the chambers 112, 114, 116 to indicate the amount of pressure within each chamber.

The sequence of signals to actuate the valves 134A, 134B, 134C are pulsed in a recurrent cycle such that one or more valves can be actuated in order to control the negative or positive pressure relative to ambient of the respective isolated pressure chambers 112, 114, 116. Preferably, the valves and chambers are sequenced such that the pressure resistance downstream or toward the heart is reduced. This should “pull” or induce blood flow toward the heart by reducing the pressure resistance load the blood must overcome, and create more space for incoming blood flow. The point of downstream should preferably be downstream of some source of resistance, such as stenosis, obstructions, faulty valves, or the like. This is advantageous over applications applying a positive pressure upstream the source of the resistance which causes a buildup of forces on the very vessel that is suffering from an inability to handle the blood flow or increased resistance.

With reference to FIG. 5, in one example of a sequence, the pressure in one chamber can be regulated to a negative pressure relative to ambient, while the other chambers have a pressure greater than the negative pressure, for example ambient or positive pressure relative to ambient. To illustrate, the valve 134C can be actuated to decrease the pressure to a negative pressure relative to ambient in only the third isolated pressure chamber 116. At the same time, the valves 134A, 134B can be actuated to increase the pressure within the first and second isolated pressure chambers 112, 114 to a pressure greater than the negative pressure of the third chamber. The pressure in the first and second isolated pressure chambers 112, 114 can be ambient or a positive pressure relative to ambient. For example, the second chamber 114 can have an ambient pressure and the first chamber 112 can have a positive pressure relative to ambient. Optionally, the first and second chambers 112, 114 can have an ambient pressure or a positive pressure, same or different, relative to ambient.

Next, the valve 134B can be actuated to decrease pressure to a negative pressure relative to ambient in only the second isolated pressure chamber 114. At the same time, the valves 134A, 134C can be actuated to increase the pressure within the first and third isolated pressure chambers 112, 116 to a pressure greater than the negative pressure of the second chamber. The pressure of the first and third isolated pressure chambers 112, 116 can then be maintained at a pressure greater than the negative pressure such as ambient or positive pressure, same or different, relative to ambient. The third chamber 116 can have an ambient pressure and the first chamber 112 can have a positive pressure relative to ambient. Optionally, the first and third chambers 112, 116 can have an ambient pressure.

Thereafter, the valve 134A can be actuated to decrease the pressure to a negative pressure in only the first isolated pressure chamber 112. At the same time, the valves 134B, 134C can be actuated to increase the pressure within the second and third isolated pressure chambers 114, 116 to a pressure greater than the negative pressure of the first chamber. The pressure within the second and third isolated pressure chambers 114, 116 can be maintained at ambient pressure. Although the third chamber is described as initially having a negative pressure, it is to be understood that the first or second chambers can initially have a negative pressure and that the sequence between the chambers can be cycled as described or in any order.

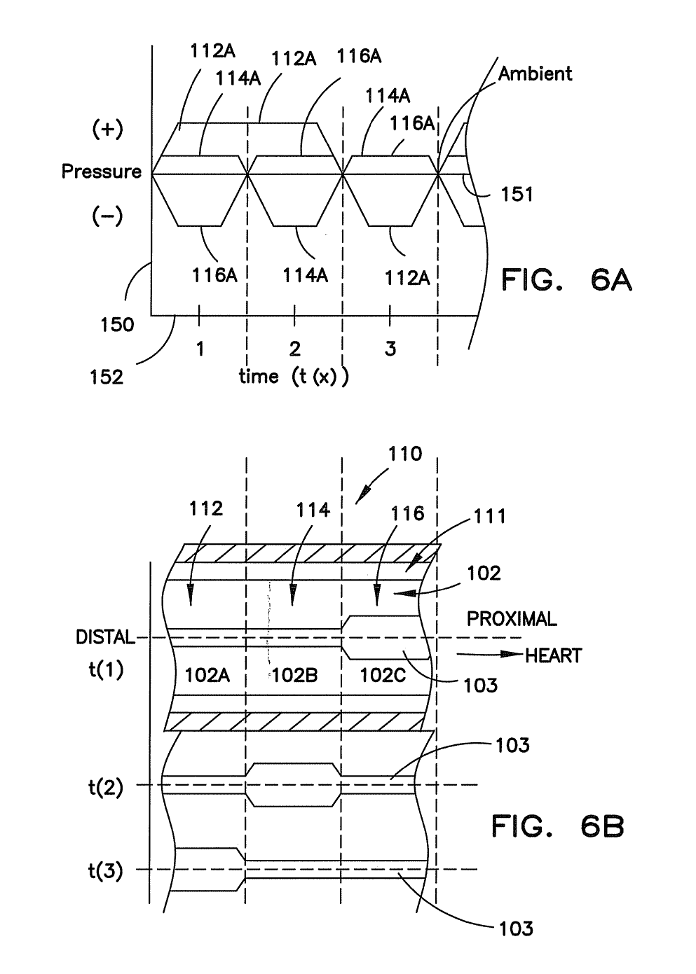

The peristaltic-like pump cycle and sequencing of the chamber pressures are depicted in FIG. 6A. This cycle is merely depicting one embodiment of a cycle with three chambers and it is to be understood that any modification to the range of pressures, the number of chambers, and the order of the dilation is within the scope of the present embodiments. The pressure 150 within each chamber can vary between a positive pressure and a negative pressure across ambient pressure 151 over time 152 in increments depicted at t1, t2, t3, etc. For example, for the third chamber 116, the pressure 116A is a negative pressure at t(1) and is increased to about ambient pressure during t(2) and t(3). For the second chamber 114, the pressure 114A is about ambient pressure at t(1), is decreased to a negative pressure at t(2), and is increased to about ambient pressure at t(3). For the first chamber 112, the pressure 112A is a positive pressure at t(1) and t(2) and is decreased to a negative pressure at t(3). Thereafter, the cycle is repeated. FIG. 6A depicts a slight offset between the pressure within a chamber and ambient for illustration purposes, although it is desirable the offset is minimized or eliminated.

FIG. 6B illustrates the effects of the peristaltic-like pump cycle and sequencing of the chamber pressures depicted in FIG. 6A on a body vessel 103 of a body part 102. Here, the body part 102 is shown within the chamber 110, which has the first, second, third isolated pressure chambers 112, 114, 116. FIG. 6B depicts the gap 111 or separation between the body part 102 and the inside wall of the chamber 110 to eliminate contact therebetween during therapy. Portions of the vessel 103 within the body part 102, corresponding to the portions 102A, 102B, 102C isolated by the chambers, are depicted being dilated at the different time increments t(1), t(2), and t(3) caused by the negative pressure environments of the chamber 110. The depicted relative size of dilation is enhanced for illustration purposes.

In another embodiment of a sequence, the pressure in two or more chambers can be regulated to a negative pressure, while the other chambers have a pressure greater than the negative pressure. For example, the valves 134B, 134C can be actuated to decrease the pressure to a negative pressure relative to ambient in only the third and second isolated pressure chambers 114, 116. At the same time, the valve 134A can be actuated to increase the pressure to a pressure greater than the negative pressures within the third and second chambers, such as ambient or positive pressure in the first isolated pressure chamber 112. Next, the valves 134A, 134C can be actuated to decrease the pressure to a negative pressure relative to ambient in only the first and third isolated pressure chambers 112, 116, while at the same time, the valve 134B can be actuated to increase the pressure to a pressure greater than the negative pressures within the first and third chambers such as ambient or positive pressure in the second isolated pressure chamber 114. Thereafter the valves 134A, 134B can be actuated to decrease the pressure to a negative pressure relative to ambient in only the first and second isolated pressure chambers 112, 114, while at the same time, the valve 134C can be actuated to increase the pressure to a pressure greater than the negative pressures of the first and second chambers such as ambient or positive pressure in the third isolated pressure chamber 116. Other sequences may include maintaining the proximal chambers at a negative pressure such that the more proximal portions of the vessel are dilated as the distal portions are being dilated. Although actuation of valve is described, it is to be understood by one skilled in the art that the valves are exemplary and the actual control of the chamber or pressure-isolated chambers can be directly controlled without valves. Pressure ranges and operable pressures within each chamber can be substantially equal within each chamber or can vary within each chamber.

The pressure of the chambers 112, 114, 116 can be timely sequenced such that the peristaltic-like pump action is maintained. The sequence can be also coordinated and/or synchronized with the pulse/heartbeat through use of sensors and the controller. For example, a sensor for sensing the blood pulse can be provided at the ankle so that when the pulse is sensed, one or more chambers can have a negative pressure relative to ambient simultaneously with, or slightly delayed from, the pulse.

The chamber 10, 110 can also be used in for better elution of bioactives to treat vascular diseases. The negative pressure, constant or fluctuating, may be beneficial to the vascular uptake of bioactives administered to the vessel via a drug eluting stent, a drug coated balloon, a weeping double balloon, or other medical devices used for local delivery of bioactives. The dilation of the vessels caused by the negative pressure increases the surface area of the luminal wall of the vessel and the spacing between cells of the wall, thereby allowing more bioactives to be uptaken such that the drug uptake efficiency is increased. Another benefit is that the increased circulatory activity will accelerate metabolism due to the simulated venous blood flow. With more blood pumping, the metabolism is accelerated which results in increased oxygen levels, which can increase the drug interactions with the vessel or tissue.

Any suitable bioactive agent can be used, and the specific bioactive agent, or bioactive agents, selected for any particular medical device according to the invention will depend upon several considerations, including the desired effect and the type of treatment and/or procedure in which the medical device is being used. Examples of suitable bioactives include heparin, covalent heparin or another thrombin inhibitor, hirudin, hirulog, argatroban, D-phenylalanyl-L-poly-L-arginyl chloromethyl ketone, or another antithrombogenic agent, or mixtures thereof; urokinase, streptokinase, a tissue plasminogen activator, or another thrombolytic agent, or mixtures thereof; a fibrinolytic agent; a vasospasm inhibitor; a calcium channel blocker, a nitrate, nitric oxide, a nitric oxide promoter or another vasodilator; an antimicrobial agent or antibiotic; aspirin, ticlopidine, a glycoprotein IIb/IIIa inhibitor or another inhibitor of surface glycoprotein receptors, or another antiplatelet agent; colchicine or another antimitotic, or another microtubule inhibitor, dimethylsulfoxide (DMSO), a retinoid or another antisecretory agent; cytochalasin or another actin inhibitor; or a remodeling inhibitor; deoxyribonucleic acid, an antisense nucleotide or another agent for molecular genetic intervention; methotrexate or another antimetabolite or antiproliferative agent; paclitaxel; tamoxifen citrate, Taxol® or derivatives thereof, or other anti-cancer chemotherapeutic agents; dexamethasone, dexamethasone sodium phosphate, dexamethasone acetate or another dexamethasone derivative, or another anti-inflammatory steroid or non-steroidal anti-inflammatory agent; cyclosporin, sirolimus, or another immunosuppressive agent; tripodal (aPDGF antagonist), angiopeptin (a growth hormone antagonist), angiogenin or other growth factors, or an anti-growth factor antibody, or another growth factor antagonist; dopamine, bromocriptine mesylate, pergolide mesylate or another dopamine agonist; ⁶⁰Co, ¹⁹²Ir, ³²P, ¹¹¹In, ⁹⁰Y, ^{99m}Tc or another radiotherapeutic agent; iodine-containing compounds, barium-containing compounds, and/or contrast agents; a peptide, a protein, an enzyme, an extracellular matrix component, a cellular component or another biologic agent; captopril, enalapril or another angiotensin converting enzyme (ACE) inhibitor; ascorbic acid, alpha tocopherol, superoxide dismutase, deferoxamine, a 21-amino steroid (lasaroid) or another free radical scavenger, iron chelator or antioxidant; a ¹⁴C—, ³H—, ¹³¹I—, ³²P— or ³⁶S-radiolabelled form or other radiolabelled form of any of the foregoing; estrogen or another sex hormone; AZT or other antipolymerases; acyclovir, famciclovir, rimantadine hydrochloride, ganciclovir sodium or other antiviral agents; 5-aminolevulinic acid, meta-tetrahydroxyphenylchlorin, hexadecaflouoro zinc phthalocyanine, tetramethyl hematoporphyrin, rhodamine 123 or other photodynamic therapy agents; an IgG2 Kappa antibody against Pseudomonas aeruginosa exotoxin A and reactive with A431 epidermoid carcinoma cells, monoclonal antibody against the noradrenergic enzyme dopamine betahydroxylase conjugated to saporin or other antibody target therapy agents; enalapril or other prodrugs; any endothelium progenitor cell attracting, binding and/or differentiating agents, including suitable chemoattractive agents and suitable polyclonal and monoclonal antibodies; cell migration inhibiting agents, such as smooth muscle cell migration inhibitors, such as Bamimistat, prolylhydrolase inhibitors, Probacol, c-proteinase inhibitors, halofuginone, and other suitable migration inhibitors; and gene therapy agents, or a mixture of any of these.

In accordance with the provisions of the patent statutes, the present invention has been described in what is considered to represent its preferred embodiment. However, it should be noted that the invention can be practiced otherwise than as specifically illustrated and described. Those skilled in the art will recognize that variations and modifications can be made without departing from the true scope and spirit of the invention as defined by the claims that follow. It is therefore intended to include within the invention all such variations and modifications as fall within the scope of the appended claims and equivalents thereof.

1. A method of treating a body vessel within a body part with a medical device, comprising:
positioning a hypobaric chamber configured to provide a negative pressure relative to ambient around a body part, the hypobaric chamber coupled to a vacuum source and including a controller coupled to the vacuum source, the controller configured to regulate the pressure within the hypobaric chamber so that a pulsating pump action is simulated within said body vessel;
varying the pressure within the hypobaric chamber between a first pressure and a second pressure to simulate said pulsating pump action, wherein the first pressure is a negative pressure relative to ambient; and
introducing said medical device within said body vessel to treat the body vessel of said body part.

2. The method of claim 1, further comprising imaging said body vessel with an imaging device to characterize the condition of the body vessel during simulation of pulsating pump action.

3. The method of claim 1, wherein the hypobaric chamber further comprises at least one glove apparatus disposed within the hypobaric chamber to provide access therein.

4. The method of claim 1, wherein the hypobaric chamber further comprises a port configured to receive the body part and a cuff disposed at the port for sealably engaging said body part.

5. The method of claim 4, wherein the hypobaric chamber further comprises an outlet port configured to receive the body part such that a portion thereof extends outwardly past the outlet port, and an impermeable article disposed at the outlet port having a cavity adapted to receive and contain the extended portion of the body part, wherein the impermeable article is configured to maintain pressure relative to ambient within the cavity.

6. The method of claim 1, wherein the hypobaric chamber further comprises two or more pressure-isolated chambers, the pressure within each pressure-isolated chamber being individually controllable.

7. The method of claim 1, wherein the medical device further comprises a bioactive, and the method further comprises maintaining a negative pressure within the chamber for a time sufficient to permit release of said bioactive to a treatment site within the body vessel.

8. A method of applying vascular therapy to a body vessel within a body part, comprising:
positioning a chamber including two or more pressure-isolated chambers around a body part, each pressure-isolated chamber separated by a partition cuff configured to sealably engage with a portion of said body part, said chamber coupled to at least one pressure source configured to provide at least one of a negative pressure and a positive pressure relative to ambient selectively within each pressure-isolated chamber of said chamber; and
decreasing the pressure within one of the pressure-isolated chambers of the pressure chamber to a negative pressure relative to ambient.

9. The method of claim 8 further comprising introducing a medical device within said body vessel to treat the body vessel.

10. The method of claim 9, wherein the medical device further comprises a bioactive, and the method further comprises maintaining said negative pressure within the chamber for a time sufficient to permit release of said bioactive to a treatment site within the body vessel.

11. The method of claim 8, wherein the two or more pressure-isolated chambers comprise a first chamber and a second chamber, and the decreasing further comprises decreasing the pressure within one of the first and second chambers to said negative pressure relative to ambient, and maintaining the pressure within the other one of the first and second chambers to a pressure greater than said negative pressure.

12. The method of claim 8, wherein the two or more pressure-isolated chambers comprise a first chamber, a second chamber, and a third chamber, the first chamber positioned distal to the second chamber and each distal to the third chamber, and the pressure within each of the chambers is controlled cyclically to simulate a peristaltic-like pump action within said body vessel.

13. The method of claim 12, wherein the decreasing further comprises decreasing the pressure within one of the first, second, and third chambers to a first negative pressure relative to ambient, then decreasing the pressure within another of the first, second, and third chambers to a second negative pressure relative to ambient, and then decreasing the pressure within the last of the first, second, and third chambers to a third negative pressure relative to ambient, and
wherein during each of the decreasing steps, the pressure within the other two chambers of the first, second, and third chambers is maintained at a pressure greater than the respective negative pressure of each chamber.

14. The method of claim 13 further comprising controlling the pressure within each of the first, second, and third chambers such that blood pressure is varied.

15. The method of claim 13 further comprising controlling the pressure within each of the first, second, and third chambers such that blood flow is varied.

16. The method of claim 13 further comprising controlling the pressure within each of the first, second, and third chambers such that blood flow is stopped.

17. The method of claim 11, wherein the decreasing further comprises decreasing the pressure within a first set of the two or more pressure-isolated chambers to a negative pressure relative to ambient, and then decreasing the pressure within a second set of two or more pressure-isolated chambers to a negative pressure relative to ambient.

18. The method of claim 8 further comprising imaging said body vessel with an imaging device to characterize the condition of the body vessel during the decreasing step.

19. A system for treating a body vessel of a body part, the system comprising:
a chamber including two or more pressure-isolated chambers, said chamber comprising a port configured to receive the body part, a first cuff disposed at the port to sealably engage with said body part, each pressure-isolated chamber separated by a second cuff configured to sealably engage with a portion of said body part,
at least one pressure source coupled to said chamber, the at least one pressure source configured to provide at least a negative pressure within each pressure-isolated chamber of said chamber, and
a controller coupled to the at least one pressure source, the controller configured to control the at least one pressure source such that the negative pressure within each of the pressure-isolated chambers is controlled cyclically to simulate a peristaltic-like pump action within said body vessel.

20. The system of claim 19, wherein the two or more pressure-isolated chambers include a first chamber and a second chamber positioned proximal to the first chamber,
the controller is further configured to control the at least one pressure source such that the pressure within the second chamber is decreased to said negative pressure relative to ambient and the pressure within the first chamber is maintained to a pressure greater than said negative pressure of the second chamber.
